(19) United States
(12) Patent Application Publication
(10) Pub. No.: US 2020/0000976 A1
(43) Pub. Date: Jan. 2, 2020

(54) A MEDICAL IMPLANT AND A METHOD OF COATING A MEDICAL IMPLANT

(71) Applicant: Field Orthopaedics Pty Ltd, Red Hill, Queensland (AU)

(72) Inventor: Christopher Arnold JEFFERY, Red Hill, Queensland (AU)

(73) Assignee: Field Orthopaedics Pty Ltd, Red Hill, Queensland (AU)

(21) Appl. No.: 16/470,188

(22) PCT Filed: Dec. 15, 2017
(86) PCT No.: PCT/AU2017/051401

§ 371 Date: Jun. 14, 2019

(30) Foreign Application Priority Data
Dec. 16, 2016 (AU) 2016905212

(51) Int. Cl. A61L 31/16; A61L 24/00; A61L 24/02; A61L 31/10

(52) U.S. Cl. CPC A61L 31/16; A61L 24/0042; A61L 24/0084; A61L 2420/08; A61L 31/10; A61L 2420/02; A61L 24/02

(57) ABSTRACT

A synthetic bead is for implantation within the body of an animal or human body. The bead includes a surface defining a shape having a bulk volume of the bead. The surface of the bead is coated with at least a first therapeutic agent to form an inner layer; and an outer layer includes a biodegradable polymer and a second therapeutic agent positioned above the inner layer.

### TECHNICAL FIELD

The present disclosure relates to a medical implant and a method of coating a medical implant.

### BACKGROUND

Any discussion of the prior art throughout the specification should in no way be considered as an admission that such prior art is widely known or forms part of the common general knowledge in the field.

Infection in surgery has always been a concern. The surgeon must cut through the protective barrier of the skin to get to the site requiring intervention. This exposes the patient and places them at risk of a deep seated infection. The incidence of infection is dependent on numerous factors from the patient's demographics and medical history, reason for the surgery and the local environment.

The complications of a surgical infection can be significant Orthopaedic periprosthetic joint infection can be a devastating, limb and life threatening condition.

The cause of infection can vary from contamination, systemic spread or emergence from an existing condition. Once bacterial colonisation in the operative site is established, the pathological process follows a fairly consistent course. The bacteria multiply using various virulent attributes to capitalise on the traumatised and poorly perfused environment, fixating on the adjacent foreign object which is the implant. The body's immune system tries to prevent this and local cells also attempt to reach the implanted material. This has been referred to as the race to the surface and is the focus of much research around infection control.

If the infection is identified and managed early enough, the bacteria fail to reach significant numbers and fail to develop an enveloping biofilm. If the biofilm is established, the infection has reached a chronic state which limits the treatment modalities available. The effect of systemic antibiotics is greatly reduced and often the only method for successful management is further surgery that involves the removal of the implant and the radical debridement of infected and devascularised tissue.

The management of bacterial infection has long focussed on the administration of effective antibiotics. In the surgical patient the specific species of bacteria and their susceptibility to antibiotics is often unknown. It is suggested that the antibiotic used should have a broad antibacterial spectrum (including gram positive and gram negative cover) and a low percentage of resistant species. The most commonly mixed antibiotics are gentamicin, tobramycin (aminoglycosides with particular effectiveness against gram-negative bacteria) and vancomycin (glycopeptide active mainly on gram-positive bacteria e.g. Staphylococcus aureus).

A crucial requirement for effective delivery of these antibiotics is reaching a concentration that can overcome the relevant bacterial ‘break point sensitivity limits_. This is the concentration that facilitates the eradication of the colony without inducing resistance to the antibiotic. One must also avoid reaching dose levels which are systemically toxic—not only eradicating the bacteria but concurrently poisoning the patient and causing cell death.

The management of infections in surgical patients, especially those with an implanted device is a specific challenge due to the added complexity of antibiotic penetration into the operative site. Antibiotic penetration is hindered by the local devascularisation and the retention of foreign material that can occur post-operative intervention. Scar creation and the formation of a new cavity can disrupt local antibiotic delivery and foreign material can facilitate the formation of a residual biofilm that can shield bacteria from antibiotics.

Current preventative options for minimising the incidence of infection associated with orthopaedic implants are associated with the implantation of antibiotic integrated composites in the space around the definitive implant. These composites come in the form of poly methyl methacrylate (medical cement), antibiotic eluting biodegradable beads or antibiotic laden polymer coatings. The limitations of these options are the structural compromise that can occur due to the presence of the beads and a limited ability to control antibiotic dosing. Dosing is compromised by the rate of dispersion either being too rapid, leading to cytotoxic concentrations or being too slow, leading to sub therapeutic doses that breed resistance.

The most common treatment approach is the use of antibiotic laden cement. The antibiotic powder is mixed into the poly methyl methacrylate (cement) manually before use. For this treatment to work the antibiotic relies on the ability to diffuse out from cracks and voids in the cement itself. The pharmacological effects of the composite are dependent on the persistence of structural defects, the viscosity of the cement, the contact surface and the concentration of antibiotic. Studies on the impact of the required voids to facilitate functional dispersion have shown a weakening of up to 36% of the structural integrity of the cement, compromising the quality of the surgery. Furthermore, even with the creation of optimal defects, due to the polymer structure and the hydrophobicity of the cement a significant portion of the antibiotic is retained and is unavailable for use. Often levels of less than 10% of the mixed antibiotic are released into the surrounding tissue and this release of drug may conclude within hours (or a few days) after surgery. Studies into the effects of dosage have shown that even with the optimal selection of cement (Palacos) and antibiotic (gentamicin and teicoplanin), at low doses very little elution occurs and at high concentrations there are local cytotoxic effects.

Studies on antibiotic effects on osteoblasts derived from trabecular bone showed that increasing gentamicin concentrations effects the function of the cells. Increasing levels of gentamicin decreased the osteoblast activity of alkaline phosphates (0 to 100 [symbol] /mL), impeding 3H-thymidine levels (>100 [symbol] /mL), and eventually inhibiting total DNA production (ℏ700 [symbol] /mL). Tobramycin at low levels (<200 [symbol] /mL) had no effect on the replication of osteoblasts, however at higher concentrations (>400 [symbol] /mL) replication decreases and eventually cell death occurs. With vancomycin, at low levels (<1.000 [symbol] /mL) there is little effect on replication, but at high concentrations (10,000 [symbol] /mL) cell death of osteoblasts occurs.

The use of antibiotic beads can be broken into the use of the traditional non-dissolvable antibiotic cement beads and the use of the newer biodegradable calcium based compounds. Cement beads function with the same mechanism as the antibiotic laden cement above, but with the added benefit of greater surface area and not being utilised for a functional role. The drawbacks of such beads is the added volume they take up in the operative cavity, the added pressure exerted by the beads within the site, and the need for removal of the beads once the infection has cleared. There is also a high potential for a locally toxic peak and a short effective time of antibiotic release. Due to these drawbacks the use of beads is also not suitable for a primary procedure or for prophylactic use. They fulfil the role of providing a first stage therapy, sterilising the field before a second stage definitive procedure. The added issue specifically with cement beads is the difficulty in locating them at the time of reconstruction and the potential for impacting mechanical performance of the definitive surgery.

The use of the dissolvable calcium sulfate beads needs special mention as they have developed a niche role in the management of infection. Calcium sulfate beads are synthetic hemihydrate calcium sulphate compounds that, like cement equivalents, are mixed with the desired antibiotic at the time of use. These calcium sulfate beads are composed of hydrophilic crystals. The hydration of these crystals from biological fluids results in the breakdown and elution of the stored antibiotic over a 2-to-3-week period. Whilst the complete breakdown of calcium sulfate beads overcomes the recollection issue of the cement alternatives they still possess the volume filling issues previously mentioned, with little data on the local concentrations or cellular effects of this method.

Therefore, there is at least a need for providing an improved way of addressing the issue of bacterial infections by preventing bacterial growth when implants are surgically placed in patients.

### SUMMARY OF THE INVENTION

In one aspect, the invention provides a medical implant comprising an implant surface, the surface comprising: an inner layer comprising a first bioceramic material and a first therapeutic agent; and an outer layer comprising a biodegradable polymer and a second therapeutic agent.

In an embodiment, the outer layer further comprises a second bioceramic material.

Preferably, the second bioceramic material is dispersed throughout the matrix of the biodegradable polymer.

In an embodiment, the biocompatible polymer is selected from the group comprising: Poly lactic acid (PLA), poly glycolic acid (PGA), Poly lactic co-glycolic acid (PLGA), and copolymers with polyethylene glycol (PEG); polyanhydrides, poly(ortho)esters, polyurethanes, poly(butyric acid), poly(valeric acid), poly(lactide-co-caprolactone) and trimethylene carbonate and combinations and co-polymers thereof.

In an embodiment, the bioceramic material is selected from the group comprising of hydroxyapatite, tricalcium phosphate, bioglass, calcium phosphate or bone or a combination thereof.

Preferably, the bioceramic material is hydroxyapatite and wherein the hydroxyapatite comprises one or more of the following ions selected from the group consisting of calcium, phosphates, fluorine, strontium, silicon and magnesium.

In an embodiment, the first therapeutic agent is adsorbed on a surface of the inner layer.

In an embodiment, the second therapeutic agent is dispersed throughout the matrix of the biodegradable polymer forming the outer layer.

In an embodiment, the first and second therapeutic agents are the same.

In an embodiment, the thickness of the outer layer is configured such that a substantial portion of the outer layer degrades under physiological conditions within a time period of 3 to 10 weeks and more preferably within a time period of 4 to 6 weeks.

Preferably, the first or second therapeutic agent is selected from the group comprising antibiotics, vitamins, chemotherapy drugs, bisphosphonates, osteoporotic drugs, growth factors, or a combination thereof.

In an embodiment, the inner layer and the outer layer is applied on the implant surface, wherein the implant preferably comprises one or materials from the group of titanium, nickel-titanium alloys, platinum-iridium alloys, gold, magnesium, stainless steel, chromo-cobalt alloys, ceramics, biocompatible plastics or polymers and combinations thereof.

In another aspect, the invention provides a synthetic bead for implantation within the body of an animal or human body, the bead comprising a surface defining a shape having a bulk volume of the bead, the bead being coated with at least a first therapeutic agent to form an inner layer; and an outer layer comprising a biodegradable polymer and a second therapeutic agent positioned above the inner layer.

In an embodiment, at least the surface of bead comprises a bioceramic material such that the first therapeutic agent is coated on the bioceramic material and wherein the bioceramic material in combination with the first therapeutic agent forms the inner layer.

In an embodiment, the outer layer further comprises a second bioceramic material.

In an embodiment, the biodegradable polymer may be selected from the group comprising: Poly lactic acid (PLA), poly glycolic acid (PGA), Poly lactic co-glycolic acid (PLGA), and copolymers with polyethylene glycol (PEG); polyanhydrides, poly(ortho)esters, polyurethanes, poly(butyric acid), poly(valeric acid), poly(lactide-co-caprolactone) and trimethylene carbonate and combinations and co-polymers thereof.

In an embodiment, the bioceramic material is selected from the group comprising of hydroxyapatite, tricalcium phosphate, bioglass, calcium phosphate or bone or a combination thereof.

In an embodiment, the bioceramic material is hydroxyapatite and wherein the hydroxyapatite comprises one or more of the following ions selected from the group consisting of calcium, phosphates, fluorine, strontium, silicon and magnesium.

In an embodiment, the first therapeutic agent is adsorbed on the surface of the synthetic bead to form the inner layer thereon.

In an embodiment, the first or second therapeutic agent is selected from the group comprising antibiotics, vitamins, chemotherapy drugs, bisphosphonates, osteoporotic drugs, growth factors, or a combination thereof.

In an embodiment, the inner layer comprises a biomimetic material with the first therapeutic agent being adsorbed on the surface of the biomimetic material.

In yet another aspect, the invention provides a bone cement for cemented arthroplasty or in the form of a drug eluting spacer implant, the bone cement comprising:
a powder component comprising:
   (a) an acrylic polymer;
   (b) a radical initiator; and
   (c) one or more synthetic beads as described herein; and

a liquid monomer component, wherein a reaction of the powder polymer component and liquid monomer component provides the bone cement composition.

In yet another aspect, the invention provides a bone void filler material for sustained release of one or more therapeutic agents, the bone void filler material comprising a biodegradable matrix having ceramic particles and synthetic beads as described herein disposed within the matrix.

In another aspect the invention provides a method of coating a medical implant, the method comprising the steps of (1) applying a bioceramic coating on a surface of an implant and contacting the bioceramic coating with a first therapeutic agent to form an inner layer; and (2) applying a biodegradable polymer and a second therapeutic agent to form an outer layer.

In an embodiment, step (2) comprises applying the biodegradable polymer and the second therapeutic agent on the inner layer to form an outer layer.

Preferably, step (2) further comprises applying the biodegradable polymer in combination with a bioceramic material.

In an embodiment, step (1) comprises adsorbing the first therapeutic agent onto a surface of the bioceramic coating.

In an embodiment, a cold plasma is disposed on the surface of the inner layer before deposition of the first therapeutic agent.

In an embodiment, the first therapeutic agent is electrostatically bonded to the bioceramic coating.

In an embodiment, formation of the inner layer in step (1) is carried out under vacuum.

In another embodiment, formation of the inner layer in step (1) is carried out under sonication, preferably pulsed-ultra-sonication.

In an embodiment, the step of applying the biodegradable polymer and the second therapeutic agent in step (2) comprises applying a solution comprising said biodegradable polymer and the second therapeutic agent.

Preferably, the solution comprises the bioceramic material, said bioceramic material being preferably dispersed in the solution.

In an embodiment, the solution is prepared by dissolving the biodegreadable polymer in the solvent, the solvent preferably being selected from acetonitrile or ethyl acetate.

In an embodiment, the second therapeutic agent is initially dissolved to form a therapeutic solution, said therapeutic solution being added to the biodegradable polymer solution.

In an embodiment of the method, the biodegradable polymer is selected from the group comprising: Poly lactic acid (PLA), poly glycolic acid (PGA), Poly lactic co-glycolic acid (PLGA), and copolymers with polyethylene glycol (PEG); polyanhydrides, poly(ortho)esters, polyurethanes, poly(butyric acid), poly(valeric acid), poly(lactide-co-caprolactone) and trimethylene carbonate and combinations and co-polymers thereof.

In an embodiment, the biodegradable polymer is a poly(lactic-co-glycolic acid) (PLGA), molar ratio 50:50, or P LGA, molar ratio 75:25, or PLGA with a free carboxyl group (PLGA-COOH), molar ratio 50:50.

In an embodiment of the method, the bioceramic material is selected from the group comprising of hydroxyapatite, tricalcium phosphate, bioglass, calcium phosphate or bone or a combination thereof.

In a preferred embodiment, the biodegradable polymer is a poly (lactic-co-glycolic acid) (PLGA) and wherein the bioceramic material is hydroxyapatite (HA).

Preferably, the PLGA is dissolved in the solvent at a concentration in the range of 0.5 w/v (%) to 40 w/v (%), more preferably 1 w/v (%) to 20 w/v (%).

In an embodiment, the HA is dispersed in the solvent at a concentration in the range of 0.1 w/v (%) to 20 w/v (%), more preferably 0.5 w/v (%) to 10 w/v (%).

In an embodiment, volumetric ratio (R) between the volume of the therapeutic solution (T) to the volume of the PLGA solution comprising dispersed HA and R ranges from about 2:8 to 5:8.

In an embodiment of the method, the solution is applied on the inner layer by air-spraying or by dip coating.

In another aspect, the invention provides a method of treating a patient in need of a medical implant, the method comprising the step of surgically placing the medical implant, as described herein, into said patent.

In another aspect, the invention provides a method of coating a synthetic bead, the synthetic bead comprising a biomimetic surface defining a shape having a bulk volume of the bead, the method comprising the following steps:

(1) coating a first therapeutic agent on the biomimetic surface to form an inner layer; and

(2) applying a biodegradable polymer and a second therapeutic agent on the inner layer to form an outer layer.

In yet another aspect, the invention also provides a method of coating a synthetic bead, the synthetic bead comprising an outer surface defining a shape having a bulk volume of the bead, the method comprising the following steps:

(1) coating a biomimetic material on the outer surface and applying a first therapeutic agent onto the biomimetic material;

(2) applying a biodegradable polymer and a second therapeutic agent on the inner layer to form an outer layer.

In an embodiment the step (2) further comprises applying the biodegradable polymer in combination with a bioceramic material.

In an embodiment, step (1) comprises adsorbing the first therapeutic agent onto a surface of the biomimetic surface.

In an embodiment, step (1) further comprises the following steps:

(a) soaking or immersing the synthetic bead in a solution comprising said first therapeutic agent for a pre-determined time period for coating the surface of the bead; and

(b) retrieving the coated synthetic beads and freeze drying said coated beads.

In an embodiment, step (2) comprises the following steps:

(c) soaking or immersing the coated beads obtained from step (1) in a solution comprising said biodegradable polymer, the second therapeutic agent and an organic solvent;

(d) evaporating the solvent from step (c) under stirring to obtain the said outer layer.

In an embodiment, step (1) comprises dissolving said first therapeutic agent in a solvent.

In an embodiment, formation of the inner layer in step (1) is carried out under vacuum.

In an embodiment, the biodegradable polymer is selected from the group comprising: Poly lactic acid (PLA), poly glycolic acid (PGA), Poly lactic co-glycolic acid (PLGA), and copolymers with polyethylene glycol (PEG); polyanhydrides, poly(ortho)esters, polyurethanes, poly(butyric acid), poly(valeric acid), poly(lactide-co-caprolactone) and trimethylene carbonate and combinations and co-polymers thereof.

In an embodiment, the biodegradable polymer is a poly(lactic-co-glycolic acid) (PLGA), molar ratio 100:0 or 90:10 or 80:20 or 75:25 or 70:30 or 65:35 or 60:40 or 50:50 or 40:60, 30:70 or 20:80 or 10:90; or PLGA, molar ratio 100:0 or 90:10 or 80:20 or 75:25 or 70:30 or 65:35 or 60:40 or 50:50 or 40:60, 30:70 or 20:80 or 10:90; or PLGA with a free carboxyl group (PLGA-C OOH), molar ratio 100:0 or 90:10 or 80:20 or 75:25 or 70:30 or 65:35 or 60:40 or 50:50 or 40:60, 30:70 or 20:80 or 10:90.

In an embodiment, the bioceramic material is selected from the group comprising of hydroxyapatite, tricalcium phosphate, bioglass, calcium phosphate or bone or a combination thereof.

In an embodiment, the biodegradable polymer is a poly(lactic-co-glycolic acid) (PLGA) and wherein the bioceramic material is hydroxyapatite (HA).

In an embodiment, the PLGA is dissolved in the solvent at a concentration in the range of 0.5 w/v (%) to 40 w/v (%), more preferably 1 w/v (%) to 20 w/v (%) and more preferably 1 w/v (%) to 10 w/v (%).

In an embodiment, the bioceramic material is dispersed in the solvent at a concentration in the range of 0.1 w/v (%) to 20 w/v (%), more preferably 0.5 w/v (%) to 10 w/v (%).

In an embodiment, the first therapeutic agent is an antibiotic agent and wherein the solution in step (1) comprises an antibiotic concentration in the range of 10% w/v to 30% w/v and more preferably in the range of 10% w/v to 25% w/v.

In an embodiment, the second therapeutic agent is an antibiotic agent and wherein the solution in step (2) comprises an antibiotic concentration in the range of 10% w/v to 30% w/v and more preferably in the range of 10% w/v to 25% w/v.

In an embodiment, a bioceramic material is dispersed in the solvent of step (c).

In an embodiment, the bioceramic material comprises one or more of the following: hydroxyapatite, tricalcium phosphate, bioglass, calcium phosphate or bone or a combination thereof.

In an embodiment, the outer layer comprises a thickness in the range of 10| m to 150| m and more preferably in the range of 20| m to 100| m.

### BRIEF DESCRIPTION OF FIGURES

FIG. 1 is a first sectional view of a medical implant 100 in accordance with a first embodiment of the present invention.

FIG. 2 is an enlarged sectional view of the medical implant 100 in accordance with the first embodiment of the present invention.

FIG. 3 is a schematic view of the medical implant 100 in accordance with the first embodiment of the present invention.

FIG. 4 is a graphical illustration showing the relationship between antibiotic elution from the medical implant 100 and time.

FIG. 5 depict results of drug elution from example 1.

FIG. 6 depicts a schematic view of a coated synthetic bead 200.

FIG. 7 depicts a schematic illustration depicting a method of coating a synthetic bead 200.

FIG. 8 depicts an enlarged schematic view of the coated synthetic bead 200.

### DETAILED DESCRIPTION

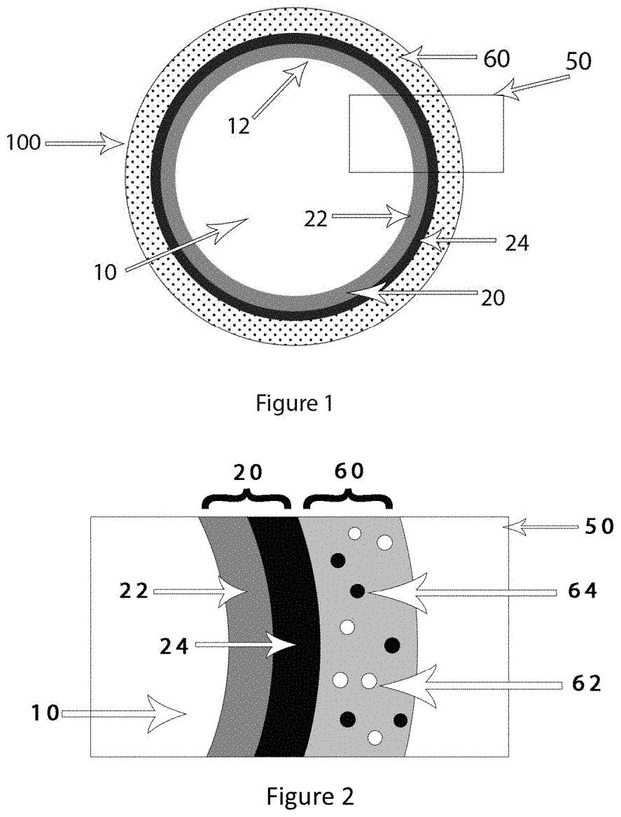

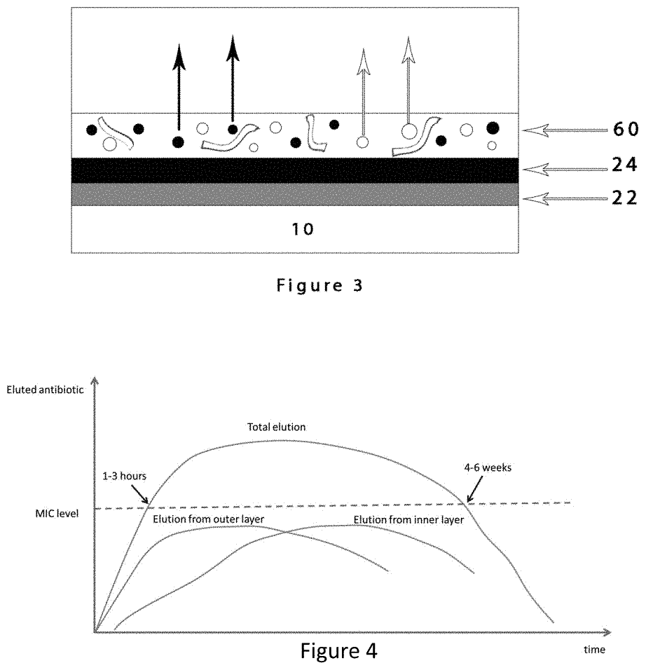

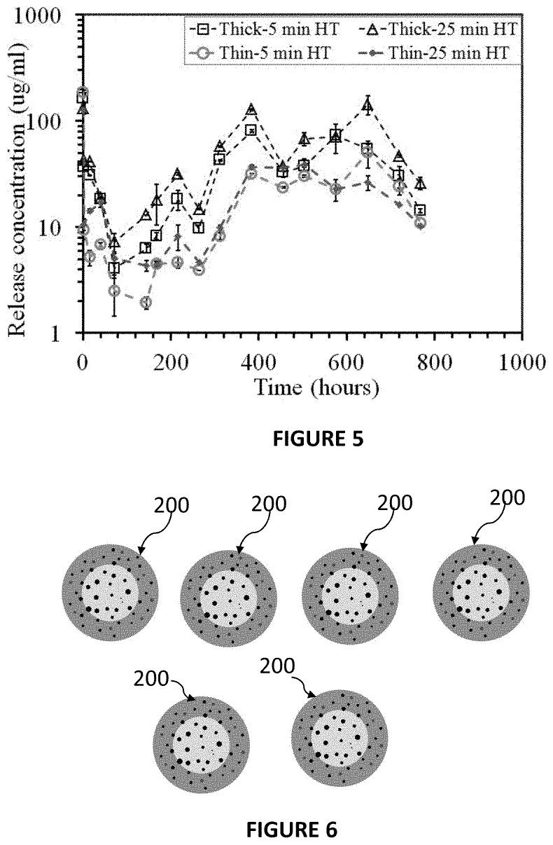

Referring to FIGS. 1 to 3, a first embodiment of a medical implant 100 in accordance with the present invention is illustrated. The implant body 10 may be formed from one or more materials from the group of titanium, nickel-titanium alloys, platinum-iridium alloys, gold, magnesium, stainless steel, chromo-cobalt alloys. The implant body 10 may also be formed from ceramic materials or polymeric materials.

In the preferred embodiment, the medical implant 100 comprises a metallic body 10 having an implant surface 12. The implant surface 12 is coated with an inner layer 20 and a second outer layer 60.

The inner layer 20 comprises a sub-layer or base layer 22 comprising biomimetic hydroxyapatite (HA) that is directly coated onto the implant surface 12 and an antibiotic coating 24 that is adsorbed on the surface of the biomimetic HA layer 22.

The outer layer 60 comprises a polymeric matrix comprising a biodegradable polymer provided by Poly lactic co-glycolic acid (PLGA) that substantially forms the outer layer 60. The outer layer 60 also comprises antibiotic particles 64 and bioceramic particles, preferably hydroxyapatite particles 62 dispersed uniformly across the matrix of the PLGA in the outer layer 60.

The medical implant 100 provides an improved coating system based on hydroxyapatite (HA) and poly (lactic-co-glycolic acid) (PLGA) that is adapted for carrying antibiotics (such as vancomycin, gentamycin). Without wishing to be bound by theory, the applicants have theorized that a coating system, in accordance with an embodiment, comprising the combination of the inner layer 20 and the outer layer 60 provides sustained elution of antibiotics over a period of 4-6 weeks and superior osteoinductivity due to the presence of biomimetic HA component in combination with the antibiotics in the inner layer 20 and the outer layer 60 in the aforementioned configuration.

Without wishing to be bound by theory, the applicants also believe that the medical implant 100 provides an improvement over previously known medical implants and coating methods for the following reasons.

The medical implant 100 having the combination of the inner layer 20 and the outer layer 60 on the implant surface 12 provides an increased antibiotic loading capacity for the medical implant 100 as will be demonstrated in the foregoing sections. Specifically, the HA layer 22 provided on the implant's surface 12 is likely to adsorb antibiotic agents 24 through physical adsorption and ionic bonding as a result of the high surface area of the HA particles on the HA layer 22 and the intrinsically high negative charge densities of the HA particles in the HA layer 22. At least some antibiotic agents such as vancomycin and gentamycin have partial positive charges under physiological pH conditions. Therefore, it is hypothesized that such positively charged antiobiotic agents are likely to be electrostatically bonded to the HA particles in the HA layer 22. The inner layer 20 is then covered by a biodegradable polymer such as PLGA to form the outer layer 60. The applicants have hypothesized that providing a biodegradable polymeric layer 60 directly above the inner layer 20 slows down drug elution, specifically elution of antibiotic agents adsorbed on the HA layer 22. Importantly, the polymer matrix of the PLGA layer 60 is formulated to contain additional dispersed antibiotic particles to provide additional loading and release during use. The co-polymer ration in the PLGA forming the PLGA layer 60 is selected such that this protective coating formed by the outer layer 60 completely degrades after 4-6 weeks in vivo. The antibiotic payload dispersed in the PLGA layer 60 is exhausted within the 4-6 weeks and biomimetic HA coating underneath is exposed to further accelerate new bone formation.

The applicants have hypothesized that the elution of antibiotic agents in the inner layer 20 and the outer PLGA layer 60 is regulated by 3 mechanisms that work together to provide sustained release of the antibiotic agent at a level above the recommended minimum inhibitory concentration (MIC) for a period of 4-6 weeks:

(a) diffusion of antibiotics agents/molecules dispersed in the outer layer 60, specifically the matrix of the PLGA 62.

(b) diffusion of antibiotics agents/molecules that are adsorbed on the HA layer 22 through the polymeric coating forming the outer layer 60 and

(c) biodegradation of the PLGA coating 62 of the outer layer 60 which takes—4-6 weeks for PLGA of 50% lactic and 50% glycolic (i.e., PLGA 50:50).

The applicants have hypothesized, as shown in FIG. 4, that the combined effects of surface diffusion, bulk diffusion and matrix erosion processes may result in antibiotic elution kinetics of desirable properties: quickly (˜2-3 hours) and locally reach therapeutic level (above MIC), remain above the MIC for extended periods of time (4-6 weeks) followed by a sharp release of the antibiotic agents (˜10-12 hrs-referred to as a ‘tail_) when drug release is completed (to avoid drug resistance development).

The applicants also believe that the medical implant 100 with the inner layer 20 and outer layer 60 provides improved osteoinductive (i.e., inducing bone formation) properties. Specifically, the outer layer 60 having the PLGA polymer matrix is formulated to contain amorphous hydroxyapatite HA particles 64 to provide additional osteoinductivity to the medical implant 100. It is understood by the applicants that that the dissolution and re-precipitation of Ca and P from HA particles in the outer layer 60 and the inner layer 20 after implantation in vivo is a major mechanism for HA to form new bone. The incorporation of amorphous HA in the outer layer 60 by incorporating HA particles 62 in the PLGA matrix of the outer layer 60. The applicants have found that higher (faster) degradation kinetics of amorphous HA particles 62 in the outer PLGA layer 60 is more favourable for bone formation.

The medical implant 100 having the combination of the inner layer 20 and the outer layer 60 on the implant surface 12 also provides increased bone ingrowth capability. Bone ingrowth largely depends on the presence of macropores. The applicants envision that, during use, bone ingrowth will not be affected by the provision of the inner layer 20 and the outer layer 60 of the medical implant 100. Preferably, the combined thickness of the inner layer 20 and the outer layer 60 will approximately be in the range of 15| m to 25| m. As a result, the combined thickness of the inner and outer layers 20 and 60 is approximately 10 times smaller than the average size of the macropores commonly found on medical implant surfaces (˜200-300 um). In addition, the amorphous HA particles 62 in the polymeric coating forming the outer layer 64 provides osteoinductivity that promote new bone formation.

The polymer coating forming the outer layer 64 also effectively shields the antibiotic agents adsorbed on the inner layer 20, specifically the antibiotic agents 24 adsorbed on the HA particles forming the HA layer 22, against excessive friction forces which may occur during insertion of certain implants.

The applicants also believe that there is an unexpected and surprising synergistic effect between the osteoinductive and biomimetic properties of the HA (provided in the first layer 20 and the second layer 60), controlled release of the antibiotic agents (24 and 64) and the biodegradable properties of PLGA in the outer layer 60. Therefore, the applicants expect that the combination of the inner layer 20 and the outer layer 60 on a medical implant is likely to provide protective effects against infection during the first critical 4-6 weeks after implant insertion and at the same time promote new bone formation and bone ingrowth into the implant surface 12 to achieve superior implant integration and reduced infection rates.

The presently described embodiment refers to antibiotic agents 24 and 64 being incorporated into the inner layer 20 and the outer layer 60. However, it is expected in alternative embodiments, therapeutic agents such as anticancer drugs (e.g., doxorubicin) or bioactive agents (e.g., BMP2) may be incorporated into the inner layer 20 or outer layer 60 without departing from the scope of the invention described herein.

A method for forming a coated medical implant 100, in accordance with another embodiment of the present invention, is described in the following sections.

In a first step, the process of forming the inner layer 20 comprises the loading of antibiotic agents 24 upon the implant surface 12 of the implant 10 coated with HA forming the HA layer 22.

In a first step, a medical implant 10 is provided. Ina second step, the implant 10 may be immersed in a simulated body fluid, such as a phosphate buffer saline (PBS) solution. The PBS solution may be prepared at various ion concentrations to mimic the chemical composition of human body fluids, such as blood plasma. The implant 10 may be initially soaked in the PBS solution and the HA coating 22 be grown biomimetically. It should be appreciated that other methods for forming the HA coating 22 may also be used in alternative embodiments.

Prior to applying the HA coating 22, a surface 12 of the implant 10 may also be coated with for example, a crystalline TiO2 coating through, for example, cathodic arc evaporation. It should be appreciated that other methods can be used to deposit a volume of the coating. The surface metal coating can be selected from the group of TiO₂, TiO, TiCrO₂, Ti₂O₃, Ti₃O₅, SiO₂, MgO₂, AlO₂, and CrO₂. In the preferred embodiment, the implant 10 may have an implant body with the implant surface 12 comprising a base metal of Ti and SST alloys. The provision of the crystalline TiO₂ coating provides a bioactive underlying surface so as to nucleate the HA crystals of the HA layer 22 on the metal base provided on the implant body 12.

The next step involves adsorbing antibiotics onto the HA-coated implant 10 obtained in the previous step.

Antibiotic powder (such as gentamycin powder) may be dissolved in aqueous solution having a pH 4.5 to 7. The HA coated implant 10 may be coated with the aqueous solution of the antibiotic powder. Before forming the antibiotic coating on the HA layer 22 of the implant 10, the HA coated implant 10 may be plasma-treated to achieve a desired charge polarization. For example, Ar-gas cold plasma may be applied for 10 minutes to create surface negative charge of about −35 mV. After the plasma treatment has created surface charge polarization desirable for strong electrostatic binding with antibiotic agents, the plasma treated implant may be immersed in the antibiotic solution.

The immersion of the plasma treated implant 10 may be followed by application of a low vacuum for 10 to 30 mins or by pulsed ultra-sonication applied for 2-5 mins to facilitate better contact between the HA coated implant 10 and the antibiotic solution to preferably achieve homogeneous antibiotic adsorption and adsorb antibiotic particles 24 on the HA layer 22. The implant 10 may be removed from the antibiotic solution and air-dried for 12 to 24 hours in the dark at room temperature. The inner layer 20 comprising the HA layer 22 with the adsorbed antibiotic particles 24 is thereby formed.

The next step involves the formation of the outer layer 60. The outer layer 60 may be formed by at least two different coating methods.

In a first alternative embodiment, the outer layer 60 comprising PLGA may be formed by way of air-drying.

Specifically, PLGA (50:50 or 75:25; MW=106 kDa) may be dissolved in a solvent such as acetonitrile or ethyl acetate at concentrations of from 1 w/v % to 20 w/v % with slight heating at 37 to 50 degree C. for 10 to 30 minutes. Amorphous hydroxyapatite (HA) powder may be dispersed into the PLGA polymer solution at a concentration from 0.5 w/v % to 10 w/v %. The PLGA solution with the HA particles dispersed in the solution may be ultrasonicated for about 30 mins to 60 mins to uniformly disperse the amorphous HA in the PLGA solution.

An antibiotic solution may be prepared by introducing antibiotic powder in an appropriate solvent (such as water, saline, PBS) at a relatively high concentration. The antibiotic solution is mixed with the PLGA solution (containing the dispersed HA particles). Specifically, the antibiotic solution is added and mixed to the HA-PLGA solution at volume ratios ranging from 2:8 to 5:8 (vol. antibiotic solution: volume HA-PLGA solution). The antibiotic⁻HA⁻PLGA solution is air-sprayed using air pressure from 1-3 bars at distance from 3.5 to 21 cm for a period of 30 seconds to 2 minutes on to the HA-coated implant rotating at speed of from 0 rpm to 60 rpm. The coated implant is air-dried at temperature from 20 to 100 degree C. for a period of 30 mins to 2 days when complete evaporation of solvents is achieved. The coated implant 10 may then be treated by a cold plasma-treatment again (for example 10 mins under Argon gas plasma) to increase hydrophilicity of the surface of the coated implant 10.

In a second alternative embodiment, the outer layer 60 comprising PLGA may be formed by way of dip-coating.

An antibiotic solution may be prepared by introducing antibiotic powder in an appropriate solvent (such as water, saline, PBS) at a relatively high concentration. The antibiotic solution is mixed with the PLGA solution (containing the dispersed HA particles). Specifically, the antibiotic solution is added and mixed to the HA-PLGA solution at volume ratios ranging from 2:8 to 5:8 (vol. antibiotic solution: volume HA-PLGA solution). The initially coated implant 10 may be dipped in the antibiotic⁻HA⁻PLGA solution. The immersion of the implant 10 may be followed by application of a low vacuum for 10 to 30 mins or by pulsed ultra-sonication applied for 2-5 mins to facilitate better contact between the inner layer 20 of the implant 10 and the antibiotic⁻HA⁻PLGA solution to form a homogeneous outer layer 60 coated on the inner layer 20. The implant 10 may be removed from the antibiotic⁻HA⁻P LGA solution and air-dried for 12 to 24 hours in the dark at room temperature.

The coated implant 100 with the outer layer 60 may once again be treated by a cold plasma-treatment again (for example 10 mins under Argon gas plasma) to increase hydrophilicity of the surface of the outer layer 60 provided on the coated implant 100.

Referring to FIGS. 6 to 8, a second embodiment of a coated synthetic bead 200 in accordance with the present invention is illustrated. Synthetic beads in the form of uniform tricalcium phosphate (TCP) porous beads 205 having an average particle size in the range of 10| m to 100| m with micro and macro pores having an outer surface 210 may be obtained or fabricated by any conventional means. In other embodiments, the synthetic beads 205 may be formed using other bio-ceramic or biomimetic materials. The outer surface 210 is coated with a base layer 215 of antibiotic solution. The porous nature of the outer surface 210 allows the antibiotic solution to be adsorbed and/or absorbed into the synthetic bead 205 thereby forming a base anti-biotic layer 215. Once the inner anti-biotic layer 215 has been formed, an outer layer 260 comprising a polymeric matrix having a biodegradable polymer provided by Poly lactic co-glycolic acid (PLGA) is formed on the base layer 215. The outer layer 260 also comprises antibiotic particles 264 and bio-ceramic particles 263 that are dispersed throughout the polymer matrix of the PLGA in the outer layer 260.

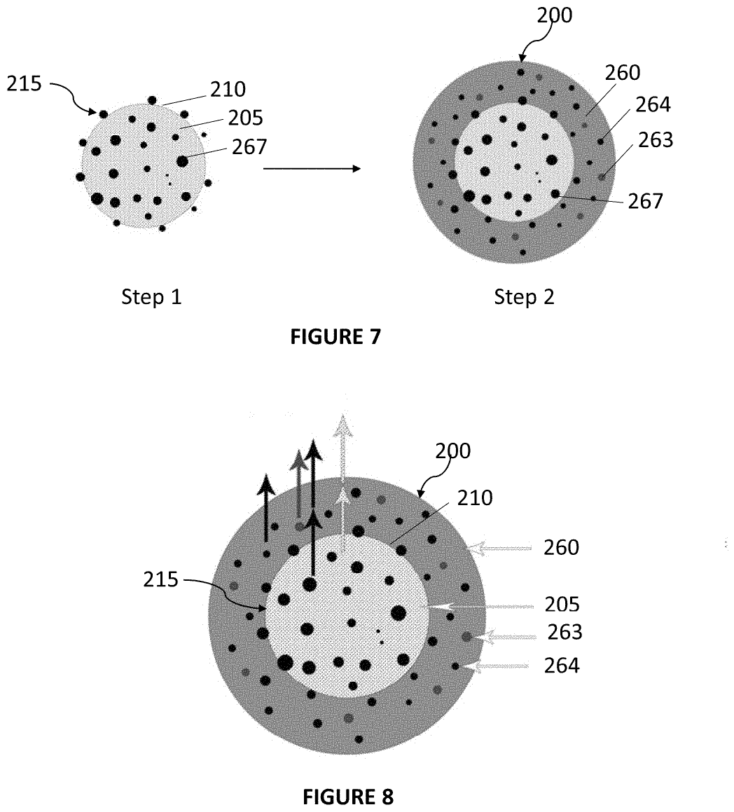

In order to form the base layer 215, as shown in Step 1 in FIG. 7, powdered antibiotic material 267 is dissolved in an appropriate solvent (e.g., water) or co-solvent and stabilizer (e.g., polyvinyl alcohol) at approximately 10-30% (wv). The TCP beads 205 are then immersed in the antibiotic solution for a period of 2-6 hours undervacuum (10⁻¹-10⁻³ Torr.) to achieve an antibiotic coating 215 on the synthetic bead 205. It is important to appreciate that the material characteristics may vary and such characteristics are expected to impact the manner in which the antibiotic material is coated on the bead 205. As shown in FIGS. 6 to 8, the porous (micro-porous or macroporous) nature of the TCP beads allows the antibiotic particles 267 to be coated not only on the outer surface 210 of the bead 205 but to also be received in the porous internal volume of the bead 205.

The method of forming the outer layer 260 once the initial antibiotic base layer 215 has been coated is illustrated in Step of FIG. 7 and explained in further detail. An antibiotic solution is formed by dissolving powdered antibiotic in an appropriate solvent (e.g., water) or co-solvent and stabilizer (e.g., polyvinyl alcohol) at approximately 10-30% (w/v). The antibiotic solution is then added into a PLGA solution (prepared at concentration of 1-10% w/v) to achieve a final antibiotic concentration in the range of 5-20% w/v.

The coated beads 205 having an initial base layer 215 are immersed in PLGA solution of 1-10% (w/v) in appropriate solvent(s) (acetone or acetonitrile or any other appropriate organic solvent) with the anti-biotic concentration of 5-20% w/v continuous stirring under low vacuum until complete evaporation of solvent(s) to form the outer layer 260 on the beads 205. In some embodiments, the outer layer 260 may be dried further by e.g., repeated spreading on glass disk with a stainless steel spatula to prevent coalescing. The thickness of the outer layer 260 may be controlled to be in the range of 20| m-100| m. The coating of the outer PLGA layer allows antibiotic particles 264 to be dispersed through the polymer matrix of the PLGA in the outer layer 260.

Bioceramic material such as hydroxyapatite or TCP particles 263 are also dispersed through the PLGA matrix of the outer layer 260.

Depending on the ratio of lactide to glycolide used for the polymerization, different forms of P LGA can be obtained: these are usually identified in regard to the molar ratio of the monomers used (e.g. PLGA 75:25 identifies a copolymer whose composition is 75% lactic acid and 25% glycolic acid). In the presently described the molar ratio in the PLGA may be 100:0, 90:10, 80:20, 75:25, 70:30, 65:35, 60:40 50:50, 40:60, 30:70, 20:80, 10:90 with molecular weight in the range of 60-134 kDa are appropriate.

The coated beads 205 provide an improved coating system based on poly (lactic-co-glycolic acid) (PLGA) that is adapted for carrying antibiotics (such as vancomycin, gentamycin). Without wishing to be bound by theory, the applicants have theorized that a coating system, in accordance with an embodiment, comprising the combination of the inner layer 215 and the outer layer 260 provides sustained elution of antibiotics over a period of 4-6 weeks and superior osteoinductivity due to the presence of biomimetic TCP component on the outer surface of the beads 205 in combination with the antibiotics in the base layer 215 and the outer layer 260 in the aforementioned configuration.

Without wishing to be bound by theory, the applicants also believe that the coated beads 205 provide an improvement over previously known synthetic beads and coating methods for the following reasons.

The coated beads 200 having the combination of the inner base layer 215 and the outer layer 260 on the surface of the synthetic bead 205 provides an increased antibiotic loading capacity for the coated synthetic beads 205 as will be demonstrated in the foregoing sections. Specifically, the outer surface of the uncoated bead comprises micorpores and/or macropores that are likely to adsorb or absorb antibiotic agents 224 through physical adsorption and ionic bonding as a result of the high surface area of the outer surface of the uncoated beads and the intrinsically high negative charge densities of the outer surface of the uncoated TCP beads. At least some antibiotic agents such as vancomycin and gentamycin have partial positive charges under physiological pH conditions. Therefore, it is hypothesized that such positively charged antiobiotic agents are likely to be electrostatically bonded to the outer surface of the TCP beads thereby forming the base layer 215. The inner base layer 215 is then covered by a biodegradable polymer such as PLGA to form the outer layer 260. The applicants have hypothesized that providing a biodegradable polymeric layer 260 directly above the inner layer 215 slows down drug elution, specifically elution of antibiotic agents adsorbed on the surface 210 of the bead.

Importantly, the polymer matrix of the PLGA layer 260 is formulated to contain additional dispersed antibiotic particles to provide additional loading and release during use. The co-polymer ration in the PLGA forming the PLGA layer 260 is selected such that this protective coating formed by the outer layer 260 completely degrades after 4-6 weeks in vivo. The antibiotic payload dispersed in the PLGA layer 260 is exhausted within the 4-6 weeks and biomimetic TCP surface having adsorbed anti-biotics in the base layer 215 is exposed to further accelerate new bone formation.

The applicants have hypothesized that the elution of antibiotic agents in the inner base layer 215 and the outer PLGA layer 260 is regulated by 3 mechanisms that work together to provide sustained release of the antibiotic agent at a level above the recommended minimum inhibitory concentration (MIC) for a period of 4-6 weeks:

(a) diffusion of antibiotics agents/molecules dispersed in the outer layer 260, specifically the matrix of the PLGA 262.

(b) diffusion of antibiotics agents/molecules in the inner base layer 215 that are adsorbed on the TCP outer surface 210 layer 22 through the polymeric coating forming the outer layer 60 and

(c) biodegradation of the PLGA coating in the outer layer 260 which takes—4-6 weeks for PLGA of 50% lactic and 50% glycolic (i.e., PLGA 50:50).

The applicants have hypothesized, that the combined effects of surface diffusion, bulk diffusion and matrix erosion processes may result in antibiotic elution kinetics in the coated synthetic beads 200 to have desirable properties: quickly (˜2-3 hours) and locally reach therapeutic level (above MIC), remain above the MIC for extended periods of time (4-6 weeks) followed by a sharp release of the antibiotic agents (˜10-12 hrs-referred to as a ‘tail_) when drug release is completed (to avoid drug resistance development).

The applicants also believe that the use of the coated beads 200 with the inner base layer 215 and outer layer 260 provides improved osteo-inductive (i.e., inducing bone formation) properties. Specifically, the outer layer 260 having the PLGA polymer matrix in some embodiments may be formulated to contain amorphous hydroxyapatite bioceramic or biomimetic particles to provide additional osteoinductivity. It is understood by the applicants that that the dissolution and re-precipitation of ions such as Ca and P from bioceramic particles such as HA particles in the outer layer 260 and the inner base layer 215 after implantation in vivo is a major mechanism for to form new bone. The applicants have found that higher (faster) degradation kinetics of amorphous HA particles in the outer PLGA layer 260 is more favourable for bone formation.

The coated beads 200 may be utilised as an added constituent in bone cement or void fillers. By way of example, the coated beads 200 may be added to a bone cement for use as a drug eluting cement in cemented arthroplasty or in the forming of a temporary drug eluting spacer implant A typical bone cement comprises a powder component comprising: an acrylic polymer (such as PMMA) and a radical initiator. The coated beads 205 may be added to the powder component of the bonce cement, before adding a liquid monomer component. The reaction of the powder component (specifically the polymer in combination with initiator) and the liquid monomer component, is accompanied by curing which provides the bone cement composition. The drug elution characteristics of the coated beads 205 are useful when use in conjunction with bone cement.

Similarly, the coated beads 200 may also be utilised for use as a constituent in bone void fillers. Typically, bone void fillers comprise a biodegradable matrix having ceramic particles. The coated beads 200 may be added to the bone void fillers to derive benefit from the improved drug elution characteristics of the aforementioned coated beads 200.

### Example 1

In an exemplary embodiment, the drug elution characteristics of the coated medical implant 100 were investigated. Specifically, elution of vancomycin and cefazolin was investigated in a dynamic, physiological resembling condition (phosphate buffer saline pH 7.4, shaking, 37 degree C.) and eluted amounts of vancomycin and cefazolin overtime were quantified using UV-visible spectrophotometry. Preliminary results have indicated eluted doses of vancomycin and cefazolin above the MIC (>0.5 microgram per ml) for 5-7 days in implant samples without coatings. Providing the inner coating 20 and the outer coating 60 is likely to extend to above 4 weeks with appropriate and thick PLGA material forming the outer coating 60.

Culture S. aureus with eluted drug showed that the drug bioactivity is preserved during preparation, loading and releasing processes. Previous in house work has shown that the binding and release of antimicrobial silver on Ti, PCL and PEEK demonstrated similar release kinetics of antimicrobial Ag for 40 days above 1 ug/mL MIC based on same release mechanisms (diffusion, degradation, erosion).

Throughout the specification, biodegradable polymers are ones which degrade to smaller fragments by enzymes present in the body. The terms ‘medical implant_, ‘implant_ and the like are used synonymously to refer to any object that is designed to be placed partially or wholly within a patients body for one or more therapeutic purposes such as for restoring physiological function, alleviating symptoms associated with disease, delivering therapeutic agents, and/or repairing or replacing or augmenting etc. damaged or diseased organs and tissues.

Representative examples of medical implants/devices include pins, fixation pins and other orthopaedic devices, dental implants, stents, balloons, drug delivery devices, sheets, films and meshes, soft tissue implants, implantable electrodes, implantable sensors, drug delivery pumps, tissue barriers and shunts. It should be appreciated that other devices listed herein are contemplated by the present disclosure.

In compliance with the statute, the invention has been described in language more or less specific to structural or methodical features. The term ‘comprises_ and its variations, such as ‘comprising_ and ‘comprised of_ is used throughout in an inclusive sense and not to the exclusion of any additional features. It is to be understood that the invention is not limited to specific features shown or described since the means herein described comprises preferred forms of putting the invention into effect. The invention is, therefore, claimed in any of its forms or modifications within the proper scope of the appended claims appropriately interpreted by those skilled in the art.

Any embodiment of the invention is meant to be illustrative only and is not meant to be limiting to the invention. Therefore, it should be appreciated that various other changes and modifications can be made to any embodiment described without departing from the spirit and scope of the invention.

1. A medical implant comprising an implant surface, the surface comprising:
an inner layer comprising a first bioceramic material and a first therapeutic agent; and
an outer layer comprising a biodegradable polymer and a second therapeutic agent.

2. A medical implant in accordance with claim 1 wherein the outer layer further comprises a second bioceramic material.

3. A medical implant in accordance with claim 2 wherein the second bioceramic material is dispersed throughout the matrix of the biodegradable polymer.

4. A medical implant in accordance with any one of the preceding claims wherein the biodegradable polymer is selected from the group comprising: Poly lactic acid (PLA), poly glycolic acid (PGA), Poly lactic co-glycolic acid (PLGA), and copolymers with polyethylene glycol (PEG); polyanhydrides, poly(ortho)esters, polyurethanes, poly(butyric acid), poly(valeric acid), poly(lactide-co-caprolactone) and trimethylene carbonate and combinations and co-polymers thereof.

5. A medical implant in accordance with any one of the preceding claims wherein the bioceramic material is selected from the group comprising of hydroxyapatite, tricalcium phosphate, bioglass, calcium phosphate or bone or a combination thereof.

6. A medical implant in accordance with any one of the preceding claims wherein the bioceramic material is hydroxyapatite and wherein the hydroxyapatite comprises one or more of the following ions selected from the group consisting of calcium, phosphates, fluorine, strontium, silicon and magnesium.

7. A medical implant in accordance with any one of the preceding claims wherein said therapeutic agent is adsorbed on a surface of the inner layer.

8. A medical implant in accordance with claim 7 wherein the therapeutic agent is dispersed throughout the matrix of the biodegradable polymer forming the outer layer.

9. A medical implant in accordance with any one of the preceding claims wherein the first or second therapeutic agent is selected from the group comprising antibiotics, vitamins, chemotherapy drugs, bisphosphonates, osteoporotic drugs, growth factors, or a combination thereof.

10. A medical implant in accordance with any one of the preceding claims wherein the inner layer and the outer layer is applied on the implant surface, wherein the implant preferably comprises one or more materials from the group of titanium, nickel-titanium alloys, platinum-iridium alloys, gold, magnesium, stainless steel, chromo-cobalt alloys, ceramics, biocompatible plastics or polymers and combinations thereof.

11. A medical implant in accordance with any one of the preceding claims wherein the inner layer comprises a biomimetic material with the first therapeutic agent being adsorbed on the surface of the biomimetic material.

12. A synthetic bead for implantation within the body of an animal or human body, the bead comprising a surface defining a shape having a bulk volume of the bead, the bead being coated with at least a first therapeutic agent to form an inner layer; and an outer layer comprising a biodegradable polymer and a second therapeutic agent dispersed in the matrix of the biodegradable polymer.

13. A synthetic bead in accordance with claim 12 wherein at least the surface of bead comprises a bioceramic material such that the first therapeutic agent is coated on the bioceramic material and wherein the bioceramic material in combination with the first therapeutic agent forms the inner layer.

14. A synthetic bead in accordance with claim 12 or 13 wherein the outer layer further comprises a second bioceramic material.

15. A synthetic bead in accordance with any one of claims 12 to 14 wherein the biodegradable polymer is selected from the group comprising: Poly lactic acid (PLA), poly glycolic acid (PGA), Poly lactic co-glycolic acid (PLGA), and copolymers with polyethylene glycol (PEG); polyanhydrides, poly(ortho)esters, polyurethanes, poly(butyric acid), poly(valeric acid), poly(lactide-co-caprolactone) and trimethylene carbonate and combinations and co-polymers thereof.

16. A synthetic bead in accordance with claim 13 or 14 wherein the bioceramic material is selected from the group comprising of hydroxyapatite, tricalcium phosphate, bioglass, calcium phosphate or bone or a combination thereof.

17. A synthetic bead in accordance with any one of claims 12 to 16 wherein the bioceramic material is hydroxyapatite and wherein the hydroxyapatite comprises one or more of the following ions selected from the group consisting of calcium, phosphates, fluorine, strontium, silicon and magnesium.

18. A synthetic bead in accordance with any one of claims 12 to 17 wherein the first therapeutic agent is adsorbed on the surface of the synthetic bead to form the inner layer thereon.

19. A synthetic bead in accordance with any one of claims 12 to 18 wherein the first or second therapeutic agent is selected from the group comprising antibiotics, vitamins, chemotherapy drugs, bisphosphonates, osteoporotic drugs, growth factors, ora combination thereof.

20. A synthetic bead in accordance with any one of claims 12 to 19 wherein the inner layer comprises a biomimetic material with the first therapeutic agent being adsorbed on the surface of the biomimetic material.

21. A bone cement for use as a drug eluting cement in cemented arthroplasty or in the forming of a drug eluting spacer implant, the bone cement comprising:
a powder component comprising:
(a) an acrylic polymer;
(b) a radical initiator; and
(c) one or more synthetic beads in accordance with any one of claims 12 to 20; and
a liquid monomer component, wherein a reaction of the powder polymer component and liquid monomer component provides the bone cement composition.

22. A bone void filler material for sustained release of one or more therapeutic agents, the bone void filler material comprising a biodegradable matrix having ceramic particles and synthetic beads in accordance with claims 12 to 20 disposed within the matrix.

23. A method of coating a medical implant, the method comprising the steps of:
(1) applying a bioceramic coating on a surface of an implant and contacting the bioceramic coating with a first therapeutic agent to form an inner layer; and
(2) applying a biodegradable polymer and a second therapeutic agent on the inner layer to form an outer layer.

24. A method in accordance with claim 23 wherein the step (2) further comprises applying the biodegradable polymer in combination with a bioceramic material.

25. A method of coating in accordance with any one of claim 23 or 24 wherein step (1) comprises adsorbing the first therapeutic agent onto a surface of the bioceramic coating.

26. A method in accordance with any one of claims 23 to 25 wherein a cold plasma is disposed on the surface of the inner layer before deposition of the first therapeutic agent.

27. A method in accordance with any one of claims 23 to 26 wherein the first therapeutic agent is electrostatically bonded to the bioceramic coating.

28. A method in accordance with any one of claims 23 to 27 wherein formation of the inner layer in step (1) is carried out under vacuum.

29. A method in accordance with any one of claims 23 to 27 wherein formation of the inner layer in step (1) is carried out under sonication, preferably pulsed-ultra-sonication.

30. A method in accordance with any one of claims 23 to 29 wherein the step of applying the biodegradable polymer and the second therapeutic agent on the inner layer in step (2) comprises applying a solution comprising said biodegradable polymer and the second therapeutic agent.

31. A method in accordance with claim 30 when dependent upon claim 30 wherein the solution comprises the bioceramic material, said bioceramic material being preferably dispersed in the solution.

32. A method in accordance with claim 30 or 31 wherein the solution is prepared by dissolving the bio-degradable polymer in the solvent, the solvent preferably being selected from acetonitrile or ethyl acetate.

33. A method in accordance with claims 30 to 32 wherein the second therapeutic agent is initially dissolved to form a therapeutic solution, said therapeutic solution being added to the biodegradable polymer solution.

34. A method in accordance with claims 23 to 31 wherein the biodegradable polymer is selected from the group comprising: Poly lactic acid (PLA), poly glycolic acid (PGA), Poly lactic co-glycolic acid (PLGA), and copolymers with polyethylene glycol (PEG); polyanhydrides, poly(ortho)esters, polyurethanes, poly(butyric acid), poly(valeric acid), poly(lactide-co-caprolactone) and trimethylene carbonate and combinations and co-polymers thereof.

35. A method in accordance with claims 23 to 34 wherein the biodegradable polymer is a poly(lactic-co-glycolic acid) (PLGA), molar ratio 50:50, or PLGA, molar ratio 75:25, or PLGA with a free carboxyl group (PLGA-COOH), molar ratio 50:50.

36. A method in accordance with claims 23 to 35 wherein the bioceramic material is selected from the group comprising of hydroxyapatite, tricalcium phosphate, bioglass, calcium phosphate or bone or a combination thereof.

37. A method in accordance with any one of claims 23 to 36 wherein the biodegradable polymer is a poly(lactic-co-glycolic acid) (PLGA) and wherein the bioceramic material is hydroxyapatite (HA).

38. A method in accordance with claim 37 when dependent upon any one of claim 34, 35 or 37 wherein the PLGA is dissolved in the solvent at a concentration in the range of 0.5 w/v (%) to 40 w/v (%), more preferably 1 w/v (%) to 20 w/v (%).

39. A method in accordance with claim 37 or claim 38 when dependent upon any one of claims 19 to 22 wherein the HA is dispersed in the solvent at a concentration in the range of 0.1 w/v (%) to 20 w/v (%), more preferably 0.5 w/v (%) to 10 w/v (%).

40. A method in accordance with any one of claim 37 or 38 when dependent upon claim 23 wherein R denotes the volumetric ratio (R) between the volume of the therapeutic solution (T) to the volume of the PLGA solution comprising dispersed HA and R ranges from about 2:8 to 5:8.

41. A method in accordance with any one of claims 30 to 40 wherein the solution is applied on the inner layer by air-spraying or by dip coating.

42. A method of coating a synthetic bead, the synthetic bead comprising a biomimetic surface defining a shape having a bulk volume of the bead, the method comprising the following steps:
(1) coating a first therapeutic agent on the biomimetic surface to form an inner layer; and
(2) applying a biodegradable polymer and a second therapeutic agent on the inner layer to form an outer layer.

43. A method of coating a synthetic bead, the synthetic bead comprising an outer surface defining a shape having a bulk volume of the bead, the method comprising the following steps:
(1) coating a biomimetic material on the outer surface and applying a first therapeutic agent onto the biomimetic material;
(2) applying a biodegradable polymer and a second therapeutic agent on the inner layer to form an outer layer.

44. A method of coating a synthetic bead in accordance with claim 42 or 43 wherein the step (2) further comprises applying the biodegradable polymer in combination with a bioceramic material.

45. A method of coating in accordance with any one of claims 42 to 44 wherein step (1) comprises adsorbing the first therapeutic agent onto a surface of the biomimetic surface.

46. A method of coating in accordance with any one of claims 42 to 45 wherein step (1) further comprises the following steps:
(a) soaking or immersing the synthetic bead in a solution comprising said first therapeutic agent for a pre-determined time period for coating the surface of the bead; and
(b) retrieving the coated synthetic beads and freeze drying said coated beads.

47. A method of coating in accordance with any one of claims 42 to 46 wherein step (2) comprises the following steps:
(c) soaking or immersing the coated beads obtained from step (1) in a solution comprising said biodegradable polymer, the second therapeutic agent and an organic solvent;
(d) evaporating the solvent from step (c) under stirring to obtain the said outer layer.

48. A method in accordance with any one of claims 42 to 47 wherein step (1) comprises dissolving said first therapeutic agent in a solvent.

49. A method in accordance with any one of claims 41 to 48 wherein formation of the inner layer in step (1) is carried out under vacuum.

50. A method in accordance with any one of claims 42 to 49 wherein the biodegradable polymer is selected from the group comprising: Poly lactic acid (PLA), poly glycolic acid (PGA), Poly lactic co-glycolic acid (PLGA), and copolymers with polyethylene glycol (PEG); polyanhydrides, poly(ortho)esters, polyurethanes, poly(butyric acid), poly(valeric acid), poly(lactide-co-caprolactone) and trimethylene carbonate and combinations and co-polymers thereof.

51. A method in accordance with any one of claims 42 to 50 wherein the biodegradable polymer is a poly(lactic-co-glycolic acid) (PLGA), molar ratio 100:0 or 90:10 or 80:20 or 75:25 or 70:30 or 65:35 or 60:40 or 50:50 or 40:60, 30:70 or 20:80 or 10:90; or PLGA, molar ratio 100:0 or 90:10 or 80:20 or 75:25 or 70:30 or 65:35 or 60:40 or 50:50 or 40:60, 30:70 or 20:80 or 10:90; or PLGA with a free carboxyl group (PLGA-COOH), molar ratio 100:0 or 90:10 or 80:20 or 75:25 or 70:30 or 65:35 or 60:40 or 50:50 or 40:60, 30:70 or 20:80 or 10:90.

52. A method in accordance with any one of claims 42 to 51 wherein the bioceramic material is selected from the group comprising of hydroxyapatite, tricalcium phosphate, bioglass, calcium phosphate or bone or a combination thereof.

53. A method in accordance with any one of claims 42 to 52 wherein the biodegradable polymer is a poly(lactic-co-glycolic acid) (PLGA) and wherein the bioceramic material is hydroxyapatite (HA).

54. A method in accordance with any one of claim 50, 51 or 53 wherein the PLGA is dissolved in the solvent at a concentration in the range of 0.5 w/v (%) to 40 w/v (%), more preferably 1 w/v (%) to 20 w/v (%) and more preferably 1 w/v (%) to 10 w/v (%).

55. A method in accordance with claim 52 wherein the HA is dispersed in the solvent at a concentration in the range of 0.1 w/v (%) to 20 w/v (%), more preferably 0.5 w/v (%) to 10 w/v (%).

56. A method in accordance with claim 46 wherein the first therapeutic agent is an antibiotic agent and wherein the solution in step (1) comprises an antibiotic concentration in the range of 10% w/v to 30% w/v and more preferably in the range of 10% w/v to 25% w/v.

57. A method in accordance with claim 47 wherein the second therapeutic agent is an antibiotic agent and wherein the solution in step (2) comprises an antibiotic concentration in the range of 10% w/v to 30% w/v and more preferably in the range of 10% w/v to 25% w/v.

58. A method in accordance with claim 47 wherein a bioceramic material is dispersed in the solvent of step (c).

59. A method in accordance with claim 56 wherein the bioceramic material comprises one or more of the following: hydroxyapatite, tricalcium phosphate, bioglass, calcium phosphate or bone or a combination thereof.

60. A method in accordance with claims 42 to 59 wherein the outer layer comprises a thickness in the range of 10| m to 150| m and more preferably in the range of 20| m to 100| m.
